(19) United States
(12) Patent Application Publication
(10) Pub. No.: US 2016/0266133 A1
(43) Pub. Date: Sep. 15, 2016

(54) NUCLEIC ACID-SCAFFOLDED SMALL MOLECULE LIBRARIES

(71) Applicants: Matthew LEVY, New Rochelle, NY (US); ALBERT EINSTEIN COLLEGE OF MEDICINE OF YESHIVA UNIVERSITY, Bronx, NY (US)

(72) Inventor: Matthew Levy, New Rochelle, NY (US)

(73) Assignee: Albert Einstein College of Medicine, Inc., Bronx, NY (US)

(21) Appl. No.: 15/032,672

(22) PCT Filed: Oct. 28, 2014
(86) PCT No.: PCT/US14/62614

§ 371 Date: Apr. 28, 2016

Related U.S. Application Data
(60) Provisional application No. 61/896,891, filed on Oct. 29, 2013.

(51) Int. Cl. G01N 33/68; C12Q 1/68; C07H 21/04

(52) U.S. Cl. CPC G01N 33/6803; C07H 21/04; C12Q 1/6804; G01N 2400/00

(57) ABSTRACT

Methods and compositions are provided for identifying novel ligands for a protein target.

### CROSS-REFERENCE TO RELATED APPLICATIONS

This application claims benefit of U.S. Provisional Application No. 61/896,891, filed Oct. 29, 2013, the contents of which are hereby incorporated by reference.

### STATEMENT OF GOVERNMENT SUPPORT

This invention was made with government support under grant number 1R21CA182330-01 awarded by the National Institutes of Health, National Cancer Institute. The government has certain rights in the invention.

### BACKGROUND OF THE INVENTION

The disclosures of all publications, patents, patent application publications and books referred to in this application are hereby incorporated by reference in their entirety into the subject application to more fully describe the art to which the subject invention pertains.

There exists mature technology for generating nucleic acid-based ligands, aptamers, which have been well-established as capture and targeting agents. Aptamers are generated by process called in vitro selection, or SELEX (the systematic amplification of ligands by exponential amplification). This is an iterative process consisting of essentially 1) an immunoprecipitation to partition away library molecules which bind a target and 2) amplification steps to regenerate the library. The cycle is typically repeated multiple times (typically 5-15) before functional molecules are identified. To date, aptamers have been selected to bind hundreds of different targets ranging from small molecules to peptides to proteins (2-4). The approach has also been used to target whole cells and has even identified aptamers which can discriminate between different cell types without prior knowledge of specific ligands (5-7). Aptamers typically bind their targets with affinities in the nanomolar to picomolar range and can have specificities on par with the best monoclonal antibodies (8). One aptamer, Macugen®, which binds the vascular endothelial growth factor, has been approved for the treatment of macular degeneration since 2004, and others are in clinical trials (9).

However, aptamers and the aptamer selection process suffer from a number of limitations which, when combined, has perhaps prevented their more widespread use. Firstly, our laboratory and others have found that aptamers are difficult to select against some protein targets. In our laboratory experience, only ˜4 of 10 proteins prove to be good targets for aptamers—a number consistent with a recently published study (10). This is perhaps not surprising when one considers the lack of chemical functionality within the 4 nucleobases. Secondly, experience over years of performing aptamer selections has demonstrated that the seemingly simple iterative selection process is often non-trivial, with multiple rounds of selection using common primer sets and hundreds of rounds of amplification often leading to the generation of artifacts which thwart the selection process. Finally, identification of winning aptamer sequences can also be non-trivial. While minimized aptamers are usually small (˜15 to 40 nucleotides), the presentation of the even smaller ‘core’ binding motif is often dependent on flanking sequence and structure. Selections are typically performed with large libraries of 70-100 nucleotides in length containing random regions of 30-60 nucleotides. Identifying the minimal aptamer sequence within the context of these non-necessary sequences to render these molecules chemically tractable often requires complex motif analysis or a series of truncation and minimization experiments placing a roadblock on high throughput production.

The present invention addresses the need for improved nucleic acid-based ligands and their selection and identification.

### SUMMARY OF THE INVENTION

This invention provides an oligonucleotide comprising a nucleotide residue comprising a modified nucleobase, wherein the modified nucleobase is a pyrimidine modified at the 5 position thereof, or a purine modified at the 7 position thereof.

Also provided is a method for identifying a ligand for a protein target comprising contacting the protein target with a plurality of any of the oligonucleotides as described herein, wherein at least two of the oligonucleotides have different sequences, subsequently washing the protein target to remove any unbound oligonucleotides of the plurality of oligonucleotides, recovering and sequencing oligonucleotides bound to the target protein, so as to thereby identify from the plurality of oligonucleotides one or more ligands for the protein target.

Also provided is a method for identifying a ligand for a protein target comprising contacting the protein target with a plurality of any of the oligonucleotides as described herein, wherein at least two of the oligonucleotides have different sequences, subsequently washing the protein target to remove any unbound oligonucleotides of the plurality of oligonucleotides, recovering and sequencing oligonucleotides bound to the target protein, counting the number of oligonucleotides of each single sequence type recovered and sequenced, and comparing the percentage of the total count of oligonucleotides counted of each single sequence type recovered and sequenced to a predetermined control percentage value determined for the plurality of oligonucleotides, wherein a single sequence type having a count percentage higher than the predetermined control percentage value is identified as a ligand for the protein target, and wherein a single sequence type having a count percentage the same as or lower than the predetermined control percentage value is identified as not being a ligand for the protein target.

Also provided is a method for identifying a ligand for a protein target comprising contacting the protein target with a plurality of oligonucleotides, wherein the oligonucleotides comprise a nucleotide residue comprising a modified phosphate group having a functional group attached thereto via a thioester bond, wherein at least two of the plurality of oligonucleotides have different sequences, subsequently washing the protein target to remove any unbound oligonucleotides of the plurality of oligonucleotides, cleaving the thioester bond to remove the functional group from the phosphate group, and recovering and sequencing oligonucleotides bound to the target protein so as to thereby identify from the plurality of oligonucleotides one or more ligands for the protein target.

Also provided is a method for identifying a ligand for a protein target comprising contacting the protein target with a plurality of oligonucleotides, wherein the oligonucleotides comprise a nucleotide residue comprising a modified phosphate group having a functional group attached thereto via a thioester bond, wherein at least two of the oligonucleotides have different sequences, subsequently washing the protein target to remove any unbound oligonucleotides of the plurality of oligonucleotides, cleaving the thioester bond to remove the functional group from the phosphate group, recovering and sequencing oligonucleotides bound to the target protein, counting the number of oligonucleotides of each single sequence type recovered and sequenced, and comparing the percentage of the total count of oligonucleotides counted of each single sequence type recovered and sequenced to a predetermined control percentage value determined for the plurality of oligonucleotides, wherein a single sequence type having a count percentage higher than the predetermined control percentage value is identified as a ligand for the protein target, and wherein a single sequence type having a count percentage the same as or lower than the predetermined control percentage value is identified as not being a ligand for the protein target.

Also provided is a plurality of any of the oligonucleotides as described herein, comprising multiple copies of oligonucleotides of a given sequence. In an embodiment of the plurality, each oligonucleotide is 10 to 20 nucleotide residues in length. In an embodiment of the plurality, each oligonucleotide comprises (i) a 5′ non-random region contiguous at its 3′ end with (ii) a random region contiguous at its 3′ end with (iii) a 3′ nonrandom region. In an embodiment of the plurality, the random region is 10 to 20 nucleotide residues in length. In an embodiment of the plurality, the oligonucleotides are from 20 to 100 nucleotide residues in length.

### BRIEF DESCRIPTION OF THE DRAWINGS

FIG. 1A-1C: Key components for the development of lectimer libraries (A) an anchor-residue, in this case a dU bearing a low affinity glycan; (B) Small structured library composed of 14 random positions; (C) different conformations for small structured library having random positions as well as primer attachment sites for sequencing. N represents the randomized region.

FIG. 2: Synthesis of pyrimidine phosphoramidites modified at the 5 position using the palladium-assisted Sonagashira cross coupling reaction. Scheme a (Ser-T): (ia)[Pd0(PPh₃)₄], CuI, Et₃N, DMF propargylacetate, rt overnight. (iia) DMT-Cl, anhy Pyridine, rt, 6 hr, (iiia) 2-cyanoethyl-N—N-diisopyopylchlorophosphoramidite, DIPEA, CH₂Cl₂, rt, 45 min. Scheme b (Phe-dC): (ib)[Pd0(PPh₃)₄], CuI, Et₃N, DMF, 4-phenylbutyne, rt overnight. (iib) acetic anhydride, DMF, rt, 20 hr. (iiib) same as (iia), (ivb) same as (iiia). Method adapted from (18).

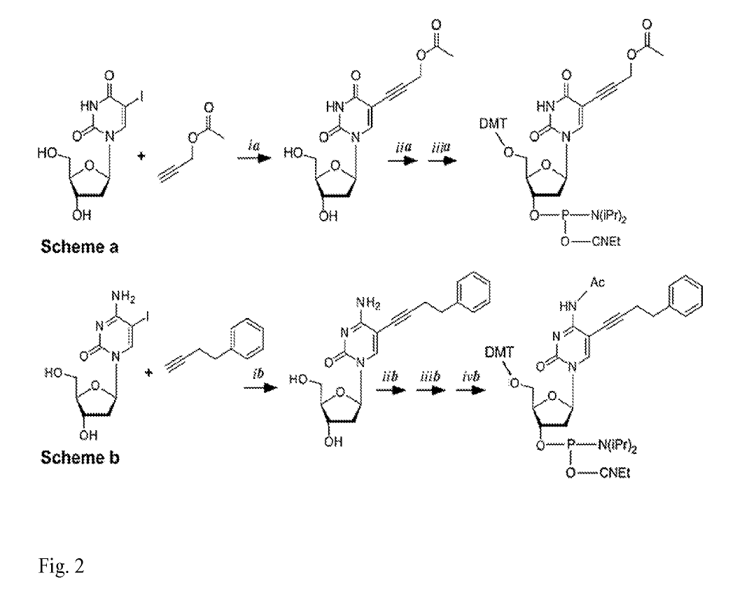

FIG. 3: Structure of modified purines to be synthesized. The terminal alkyne derivative of 4-aminobenzonitrile or 4-phenoxyaniline will be generated via reaction with propolic acid. The modified purines can be synthesized from the corresponding 7-deaza-7-iodo purine as previously described (1).

FIG. 4A-4B: (A) Modified libraries are amplifiable by standard PCR. (B) Sequencing analysis of modified libraries showing distribution in the random region.

FIG. 5: Preferred positions for R group modifications of modified nucleotides.

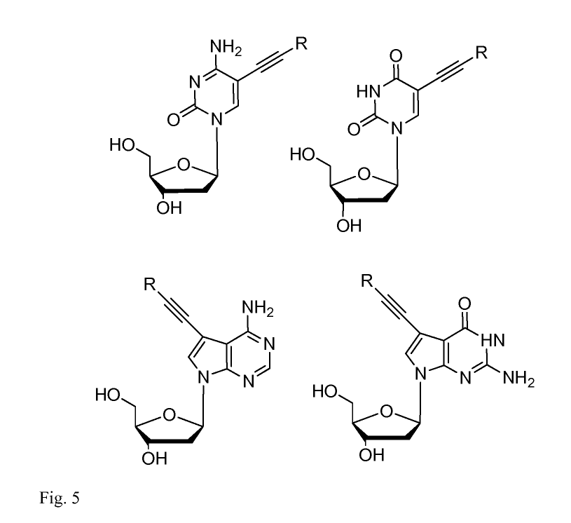

### DETAILED DESCRIPTION OF THE INVENTION

This invention provides an oligonucleotide comprising a nucleotide residue comprising a modified nucleobase, wherein the modified nucleobase is a pyrimidine modified at the 5 position thereof, or a purine modified at the 7 position thereof.

In an embodiment of an oligonucleotide of the invention, the modified nucleobase is a pyrimidine modified at the 5 position thereof with one of:

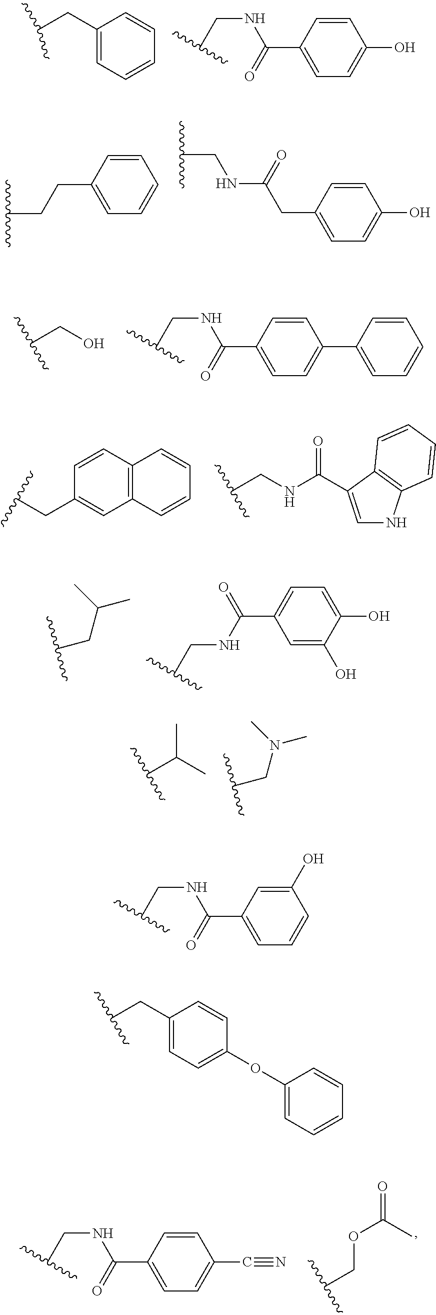

or wherein the modified nucleobase is a purine modified at the 7 position thereof with one of:

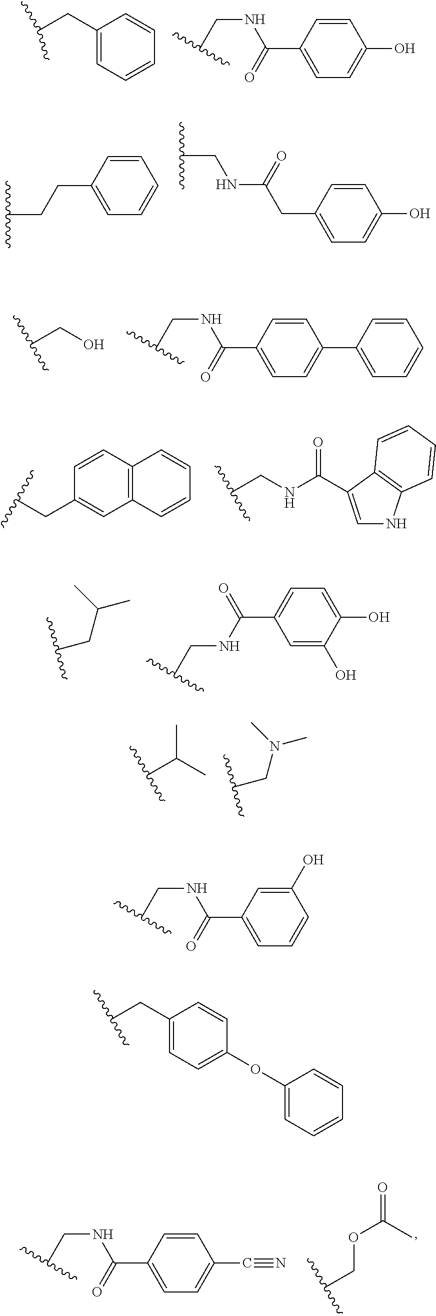

wherein the wavy line in the structures represents the point of attachment of the modifying group to the base of the modified nucleotide residue.

In an embodiment of an oligonucleotide of the invention, the modifying group is attached via an alkyne to the base of the modified nucleotide residue.

In an embodiment of an oligonucleotide of the invention, the nucleotide residue comprising a modified nucleobase comprises a deoxyuridine or a deoxycytidine or a deoxyadenine or a deoxyguanosine.

In an embodiment of an oligonucleotide of the invention, the nucleotide residue comprising a modified nucleobase comprises one of the following structures:

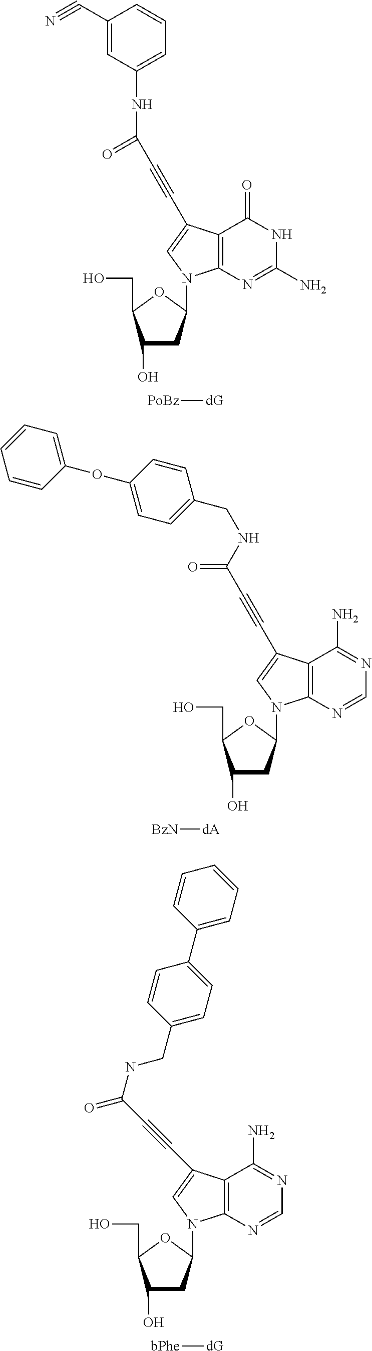

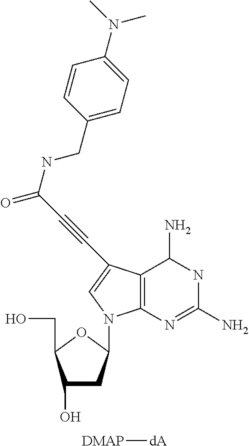

wherein each of the OH groups on the deoxyribose are, optionally, replaced with an internucleotide phosphodiester bond when the residue is not a terminal residue within the oligonucleotide.

In an embodiment of an oligonucleotide of the invention, the nucleotide residue comprising a modified nucleobase comprises one of the following structures:

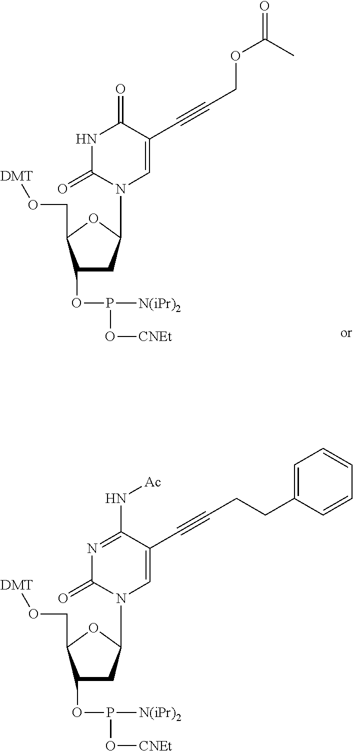

wherein each of the DMT and CNEt groups on the deoxyribose are, optionally, replaced with a further nucleotide, via an internucleotide phosphodiester bond, when the residue is not a terminal residue within the oligonucleotide. In an embodiment, the 2′ position on the sugar is an —H. In an embodiment, the 2′ position on the sugar is an —OH. In an embodiment, the 2′ position is modified to be a —OMe, —F or —NH₃. In an embodiment, the 2′ position is not modified and is —H or —OH.

In an embodiment of an oligonucleotide of the invention, the oligonucleotide comprises more than one nucleotide residue comprising a modified nucleobase, wherein the modified nucleobases are each independently chosen from: a pyrimidine modified at the 5 position thereof and a purine modified at the 7 position thereof.

In an embodiment of an oligonucleotide of the invention, the oligonucleotide comprises at least two different modified nucleobases.

In an embodiment of an oligonucleotide of the invention, the oligonucleotide comprises at least three different modified nucleobases.

In an embodiment of an oligonucleotide of the invention, the oligonucleotide comprises at least four different modified nucleobases.

In an embodiment of an oligonucleotide of the invention, the oligonucleotide comprises further a predefined ligand for a protein target attached thereto. In an embodiment, the predefined ligand is a known ligand for the protein target. In an embodiment, the predefined ligand for a protein target the predefined ligand is a low affinity ligand for the protein target. In an embodiment, the low-affinity ligand is a glycan. In an embodiment, “low affinity” means at least single digit μM to mM affinity (e.g. single digit or greater Kd).

In an embodiment of an oligonucleotide of the invention, the oligonucleotide comprises the following residue:

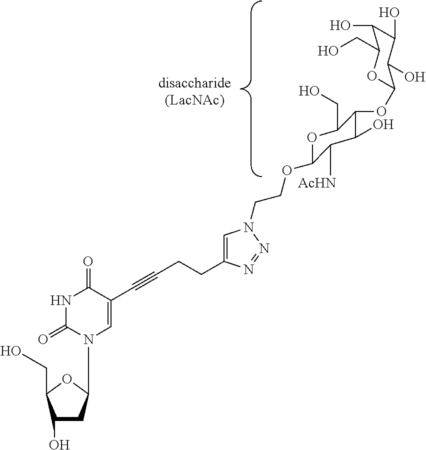 [SMILES: CC(=O)NC1C(OCCn2cc(CCC#Cc3cn([C@H]4C[C@@H](O)[C@@H](CO)O4)c(=O)[nH]c3=O)nn2)OC(CO)C(OC2OC(CO)C(O)C(O)C2O)C1O]

wherein each of the OH groups on the deoxyribose are, optionally, replaced with an internucleotide phosphodiester bond when the residue is not a terminal residue within the oligonucleotide. In an embodiment, the 2′ position on the sugar is an —H. In an embodiment, the 2′ position on the sugar is an —OH. In an embodiment, the 2 position is modified to be a —OMe, —F or —NH₃. In an embodiment, the 2′ position is not modified and is —H or —OH.

In an embodiment of an oligonucleotide of the invention, the oligonucleotide comprises a predefined ligand for a protein target attached through a functional group attached to a nitrogenous base of a nucleotide thereof.

In an embodiment of an oligonucleotide of the invention, the oligonucleotide is artificially synthesized.

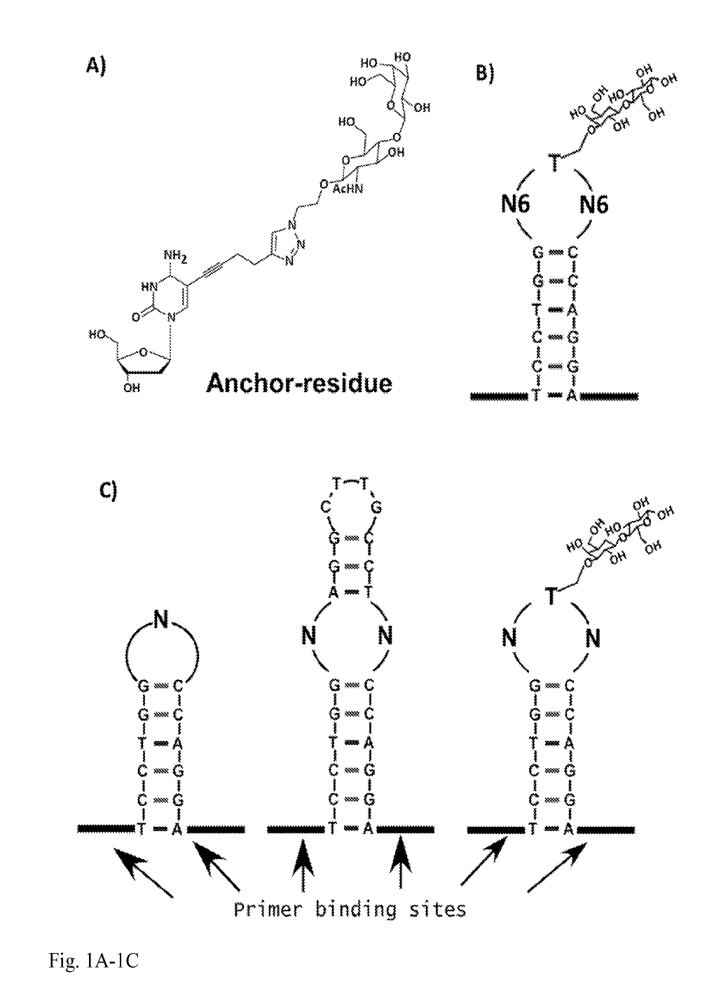

In an embodiment of an oligonucleotide of the invention, the oligonucleotide comprises (a) (i) a 5′ non-random region contiguous at its 3′ end with (ii) a random region contiguous at its 3′ end with (iii) a 3′ non-random region; or (b) (i) a 5′ non-random region contiguous at its 3′ end with (ii) a random region contiguous at its 3′ end with (iii) a second non-random region contiguous at its 3′ end with (iv) a second random region contiguous at its 3′ end with (v) a 3′ non-random region. Non-limiting examples are set forth in FIGS. 1B-1C.

In an embodiment of an oligonucleotide of the invention, the oligonucleotide comprises one or more primer attachment sequences in a non-random region thereof. In an embodiment, the one or more primers are universal primers.

In an embodiment of an oligonucleotide of the invention, the oligonucleotide comprises one or two double-stranded regions composed of intra-oligonucleotide base pairing.

Also provided is a method for identifying a ligand for a protein target comprising contacting the protein target with a plurality of any of the oligonucleotides as described herein, wherein at least two of the oligonucleotides have different sequences, subsequently washing the protein target to remove any unbound oligonucleotides of the plurality of oligonucleotides, recovering and sequencing oligonucleotides bound to the target protein, so as to thereby identify from the plurality of oligonucleotides one or more ligands for the protein target.

In an embodiment, the method further comprises counting the number of oligonucleotides of a single sequence type recovered and sequenced, wherein an oligonucleotide with the greatest count is identified as the most efficacious ligand for the protein target.

Also provided is a method for identifying a ligand for a protein target comprising contacting the protein target with a plurality of any of the oligonucleotides as described herein, wherein at least two of the oligonucleotides have different sequences, subsequently washing the protein target to remove any unbound oligonucleotides of the plurality of oligonucleotides, recovering and sequencing oligonucleotides bound to the target protein, counting the number of oligonucleotides of each single sequence type recovered and sequenced, and comparing the percentage of the total count of oligonucleotides counted of each single sequence type recovered and sequenced to a predetermined control percentage value determined for the plurality of oligonucleotides, wherein a single sequence type having a count percentage higher than the predetermined control percentage value is identified as a ligand for the protein target, and wherein a single sequence type having a count percentage the same as or lower than the predetermined control percentage value is identified as not being a ligand for the protein target.

In an embodiment, the method further comprises determining the control percentage value determined for the plurality of oligonucleotides for each sequence type.

In an embodiment of the methods, sequencing is performed subsequent to amplifying the number of the recovered sequences.

In an embodiment, the methods further comprise cleaving the modified pyrimidine at the 5 position thereof, or the modified purine at the 7 position thereof to remove the modifying group prior to amplification of the recovered sequences.

Also provided is a method for identifying a ligand for a protein target comprising contacting the protein target with a plurality of oligonucleotides, wherein the oligonucleotides comprise a nucleotide residue comprising a modified phosphate group having a functional group attached thereto via a thioester bond, wherein at least two of the plurality of oligonucleotides have different sequences, subsequently washing the protein target to remove any unbound oligonucleotides of the plurality of oligonucleotides, cleaving the thioester bond to remove the functional group from the phosphate group, and recovering and sequencing oligonucleotides bound to the target protein so as to thereby identify from the plurality of oligonucleotides one or more ligands for the protein target.

Also provided is a method for identifying a ligand for a protein target comprising contacting the protein target with a plurality of oligonucleotides, wherein the oligonucleotides comprise a nucleotide residue comprising a modified phosphate group having a functional group attached thereto via a thioester bond, wherein at least two of the oligonucleotides have different sequences, subsequently washing the protein target to remove any unbound oligonucleotides of the plurality of oligonucleotides, cleaving the thioester bond to remove the functional group from the phosphate group, recovering and sequencing oligonucleotides bound to the target protein, counting the number of oligonucleotides of each single sequence type recovered and sequenced, and comparing the percentage of the total count of oligonucleotides counted of each single sequence type recovered and sequenced to a predetermined control percentage value determined for the plurality of oligonucleotides, wherein a single sequence type having a count percentage higher than the predetermined control percentage value is identified as a ligand for the protein target, and wherein a single sequence type having a count percentage the same as or lower than the predetermined control percentage value is identified as not being a ligand for the protein target.

In an embodiment, the methods further comprise determining the control value determined for the plurality of oligonucleotides for each sequence type.

In an embodiment of the methods, sequencing is performed subsequent to amplifying the number of the recovered sequences.

In an embodiment of the methods, one or more of the plurality of the oligonucleotides comprise a nucleotide residue comprising a modified phosphate group having a functional group attached thereto via a thioester bond having the following structure:

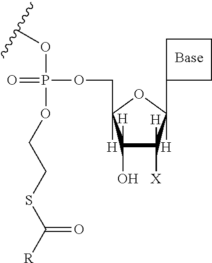

wherein the single wavy line represents the point of attachment through a phosphodiester bond to a 5′ nucleotide residue in the oligonucleotide relative to the nucleotide residue comprising a modified phosphate group shown and wherein the double wavy line represents the point of attachment through a phosphodiester bond to a 3′ nucleotide residue in the oligonucleotide relative to the nucleotide residue comprising a modified phosphate group shown, except for the situation where the nucleotide residue comprising a modified phosphate group as shown is the 5′ terminal residue or the 3′ terminal residue, respectively,

and wherein R is a chemical functional group and wherein the X at the 2′ position of the sugar is an H if the oligonucleotide is an oligodexoynucleotide, and wherein the X at the 2′ position of the sugar is an OH if the oligonucleotide is an oligoribonucleotide, or the X at the 2′ position is modified to be a —OMe, —F or —NH₃. In an embodiment, the X at 2′ position is not modified and is —H or —OH as follows:

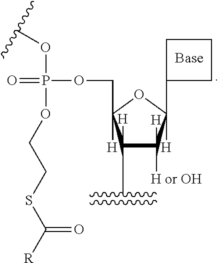

In an embodiment of the methods, the oligonucleotide is an oligodexoynucleotide.

In an embodiment of the methods, the oligonucleotide is an oligoribonucleotide.

In an embodiment of the oligonucleotide, the oligonucleotide is an oligodexoynucleotide.

In an embodiment of the oligonucleotide, the oligonucleotide is an oligoribonucleotide.

Also provided is a plurality of any of the oligonucleotides as described herein, comprising multiple copies of oligonucleotides of a given sequence. In an embodiment of the plurality, each oligonucleotide is 10 to 20 nucleotide residues in length. In an embodiment of the plurality, each oligonucleotide comprises (i) a 5′ non-random region contiguous at its 3′ end with (ii) a random region contiguous at its 3′ end with (iii) a 3′ nonrandom region. In an embodiment of the plurality, the random region is 10 to 20 nucleotide residues in length. In an embodiment of the plurality, the oligonucleotides are from 20 to 100 nucleotide residues in length.

In an embodiment of the plurality, one or more oligonucleotides of the plurality comprise a predefined ligand for a protein target, which ligand is attached through a functional group attached to a nitrogenous base of a nucleotide thereof of the random region of an oligonucleotide. In an embodiment of the plurality, the predefined ligand for the protein target is a low-affinity ligand for the protein target.

In an embodiment, the predefined ligand for the target is a sugar, a small molecule, a peptide a cytokine or another protein. Non-limiting examples of small molecule predefined ligands encompassed by the invention are folate, a folate analog, a nucleoside analog, a taxane. Non-limiting examples of peptide predefined ligands encompassed by the invention are an RGD peptide or a recognition sequence for an integrin.

In an embodiment, the predefined ligand is a low-affinity ligand. In an embodiment, the low-affinity ligand is a sugar. In different embodiments, the sugar is a monosaccharide, a disaccharide, a trisaccharide or a tetrasaccharide. Non-limiting examples of low-affinity ligands encompassed by the invention are LacNac, GalNac, Galactose, Maltose, Dextrose, Lewis X, Lewis Y, Sialyl-Lewis A, Lactose, Xylose, glucose, and sialic acid.

In an embodiment of the invention, the modified nucleobase is a modified A, U, G, C or T.

In an embodiment of the methods, the sequencing is performed by massively parallel signature sequencing, polony sequencing, pyrosequncing (for example, 454), dye sequencing (for example Illumina), SOLiD sequencing, Ion Torrent semiconductor sequencing (using hydrogen ion detection), DNA nanoball sequencing, Heliscope single molecule sequencing, Single molecule real time (SMRT) sequencing.

In an embodiment of the methods, the sequencing is performed by Nanopore DNA sequencing, Tunnelling currents DNA sequencing, Tunnelling currents DNA sequencing, Sequencing by hybridization using a DNA microarray, Sequencing with mass spectrometry, Microfluidic Sanger sequencing, Microscopy-based techniques, RNAP sequencing, or in vitro virus high-throughput sequencing.

In an embodiment of the methods employing sequencing, only one round of sequencing is employed.

In an embodiment, the oligonucleotide(s) is/are one of 10, 11, 12, 13, 14, 15, 16, 17, 18, 19, 20, 21, 22, 23, 24, or 25 nucleotide residues in length. Each individual length is an embodiment of the invention. In an embodiment, the random portion of the oligonucleotide(s) described herein is one of 10, 11, 12, 13, 14, 15, 16, 17, 18, 19, 20, 21, 22, 23, 24, or 25 nucleotide residues in length. Each individual length of the random portion is an embodiment of the invention. Total lengths of these oligonucleotides of 20 through 100 nucleotides are encompassed. Each individual integer in the series 20 through 100 as the total length is an embodiment of the invention.

An analogous approach can be applied to other sugar backbones, such as 2′ F, 2′ NH₃ or 2′ OMe.

The phrase “and/or” as used herein, with option A and/or option B for example, encompasses the individual embodiments of (i) option A alone, (ii) option B alone, and (iii) option A plus option B.

It is understood that wherever embodiments are described herein with the language “comprising,” otherwise analogous embodiments described in terms of “consisting of” and/or “consisting essentially of” are also provided.

Where aspects or embodiments of the invention are described in terms of a Markush group or other grouping of alternatives, the present invention encompasses not only the entire group listed as a whole, but each member of the group subjectly and all possible subgroups of the main group, but also the main group absent one or more of the group members. The present invention also envisages the explicit exclusion of one or more of any of the Markush group members in the claimed invention.

All combinations of the various elements described herein are within the scope of the invention unless otherwise indicated herein or otherwise clearly contradicted by context.

In the event that one or more of the literature and similar materials incorporated by reference herein differs from or contradicts this application, including but not limited to defined terms, term usage, described techniques, or the like, this application controls.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

A novel platform technology is disclosed herein which leverages the structural rigidity of nucleic acids and the ease with which they can be amplified and characterized molecularly (sequenced) with the enhanced chemical functionality observed in peptides, proteins and small molecular drugs. To achieve this goal, libraries of small molecules attached to a nucleic acid scaffold are generated. The small molecules are positioned such that they do not interfere with the ability of the nucleic acid scaffold to serve as a faithful template for polymerases (except for an embodiment of the invention where the functional groups of bound aptamer ligands are cleaved prior to amplification and sequencing, in which case a wider range of attachment points on the nucleotide residue are available). In this way, the identity of individual molecules in the library can be directly read out by sequencing, being known beforehand which sequences comprise which modifications. As such, all, one or a subset of each type of nucleobase can be modified in a given sequence as long as the positions of said modifications are predefined (e.g. through chemical synthesis). The scaffolded libraries are generated synthetically and subsequently utilized in a selection scheme coupled with next generation sequencing (NGS) which is capable of generating up to 3×10⁹ independent reads per chip. In this way, using only traditional statistics and/or motif analysis, functional variants can be readily identified within the population in only a single round by sequencing. Ligands for the target as identified though this approach can be re-synthesized using standard solid-phase DNA/RNA synthesis and further assayed for function if desired.

There are two main variations on this approach. The first makes use of just the scaffolded libraries. The second describes a similar approach but incorporates a known low affinity ligand as part of an ‘anchor residue,’ for example, the sugar lactose.

Work from Eaton and others has demonstrated that by augmenting aptamer libraries with uridine residues bearing hydrophobic modifications such as isobutyl, benzyl or tryptophanyl, the ‘hit rate’ for the identification of aptamers which target proteins could be dramatically improved (10,11). A company, Somalogic, uses certain modified uredines to perform selections using the basic SELEX method which involves multiple rounds of selection to identify modified aptamers for a diagnostic platform. The addition of a chemical modification to the library not only increases the hit rate but also results in molecules with much lower binding constants, typically in the pM range (10,11). Using a very different approach, the Liu lab has recently developed a ‘synthetic translation’ approach to generating combinatorial libraries of ssDNA bearing a variety of chemical functional groups. In this approach, a series of short oligonucleotides 10 nucleotides in length were generated synthetically and attached to one of 8 different small molecules. These short oligonucleotides were subsequently assembled by ligation into a library composed of ˜10̂6 longer oligomers ˜100 nucleotides in length which displayed up to 10 different functional groups. Libraries were then used in a SELEX style selection scheme to identify inhibitors of the enzyme carboxyanhydrase.

Advances in sequencing approaches such as next generation sequencing (NGS) have been applied to aptamer selections and offer the promise of shortening the timescale for the selection process to a few rounds of selection (13,14).

The novel approach herein eliminates the paucity of chemical functionality in nucleic acids through the use of multiple functionalized nucleotides and can avoid the multiple cycles require by the traditional selection process through the use of one round, NGS-coupled SELEX. The resulting high-throughput method can rapidly identify and validate affinity reagents that have the ease of synthesis of nucleic acids, but with an increased range of chemical functionality and binding potential. The novel ligands' function likely relies on how the combination of side groups chosen for a library are arranged and displayed on the DNA backbone. Libraries of small molecules are generated which are displayed on a nucleic acid backbone with, for example, up to four different kinds of functional groups, one on each base. Modifications are preferably positioned such that they do not interfere with the ability of these nucleic acids to base pair or serve as faithful templates for replication by polymerases. Importantly, unlike previous approaches in which base modified nucleotides have been enzymatically incorporated into DNA, RNA or into aptamer libraries (10,11,17), the libraries are generated synthetically thus allowing for a diverse array of modifications. Synthesis permits easy incorporation of multiple modifications simultaneously into a single library. Thus, the identity and variety of modifications are not limited to modifications which can be tolerated as substrates for polymerases (17). It is preferable that these modifications do not interfere with the ability of these nucleic acids to serve as faithful templates for replication by polymerases, a much easier task. However, in an alternative embodiment, the modifications of the modified aptamers that bound the target can be cleaved off prior to amplification to permit subsequent sequencing to identify the oligonucleotide.

While enhancing the chemical diversity of nucleic acid libraries with 4 additional functional groups might still seem somewhat chemically ‘limited’ as described above, even the addition of a single additional functional group has been shown to dramatically enhance the function of aptamers libraries. Moreover, different functional groups can be added to the same type of base (for example, different occurrences of a deoxycytidine) in an oligonucleotide as long as it is predetermined which functional group is attached to the nucleobase at a given position in the oligonucleotide. Additionally, this will allow use of smaller libraries, which will not only abet the selection process but facilitate downstream use by obviating the need for minimization.

An alternative approach to this methodology is disclosed in which modified libraries comprise oligonucleotides each ‘anchored’ with a low affinity ligand to a known target. For example, carbohydrate binding proteins typically bind individual sugars with low affinity (i.e. Kd=single digit or more μM to mM) with high affinity garnered though multivalent interactions between the protein and multiple copies of the target sugar(s) typically displayed in linear or branched chains (18,19). Nucleic acid ‘scaffold libraries’ are generated which display a specific ‘anchor’ sugar that possesses some basal affinity (μM to mM) for the target protein carbohydrate binding protein(s) (FIG. 1A). The anchor residue is placed at a predetermined site within the random region of the library (FIG. 1B) which will be generated using non-natural chemically functionalized nucleic acids. In this way, both the structure of the nucleic acid backbone and the appended chemical moieties work in concert to generate additional contacts to convert the initial low affinity ligand into a high affinity interaction with high specificity to the target protein.

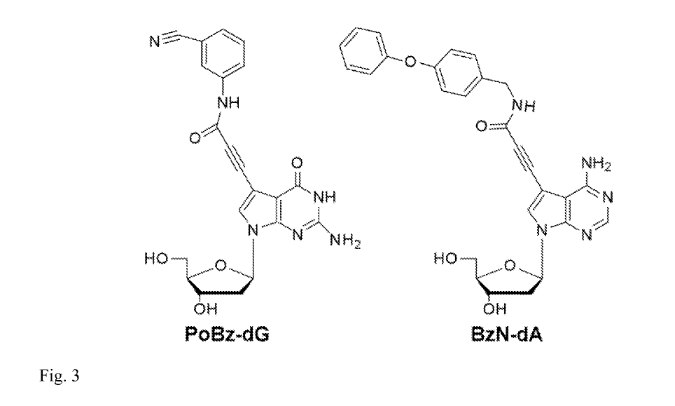

Monomer synthesis: A deoxycytidine (dC) variant was synthesized bearing a benzyl ring (Phe-dC) as well as a deoxyuridine (dU) variant bearing a hydroxyl group (SerdU) appended to the 5 position of these bases by an alkyne (20). The methods have proven to be straightforward and proceed to high yield (>80%) for each step. Functional groups readily available as terminal alkynes are preferred, incorporated using Sonogashira cross coupling, compatible with solid phase DNA/RNA synthesis and those which mimic amino acid functional groups which are not otherwise available in DNA. For example, the introduction of phenylalanine provides a hydrophobic moiety that, unlike the bases themselves (A and G possess significant hydrophobic character), is more free of the constraints imposed by the deoxyribose backbone and the drive towards base pairing. Indeed, a recent crystal structure of an anti-thrombin aptamer revealed 5 unpaired adenine residues essential in making contacts with the protein (21). Nucleotide phosphoramidites can be used to make oligonucleotides and libraries bearing single and double modifications. Modified purines are synthesized in a similar manner (also see Carell et al. (1)), for example bearing two additional, non-biological functional groups that are often found in small molecule drugs. Using this approach, for example, a deoxyadenine (dA) variant can be generated bearing an unnatural benzonitrile group (FIG. 3; BzN-dA) and a deoxyguaninidine(dG) variant can be generated bearing a 4-phenoxybenzenyl group (FIG. 3; PoBz-dG). Other modifications are possible.

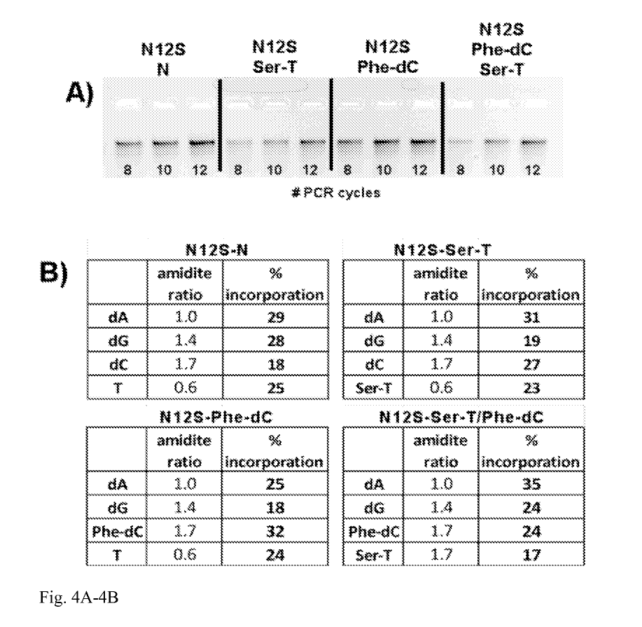

Library synthesis and characterization: The phosphoramidites developed were initially used to generate control oligonucleotides to ensure equal and efficient incorporation and the ability to serve as faithful templates for amplification. Chemical incorporation into the oligonucleotides can be confirmed by nuclease digestion, HPLC and mass spectrometry as previously described (1,22). Primer extension assays were performed to ensure that the incorporation of the modified base did not interfere with template function. A small N12 library was made containing the Phe-dC and Ser-T phosphoramidites individually or together. The single-stranded DNA libraries were amplified by PCR and compared with a library which contained no modifications (FIG. 4A). A sequence analysis of the random regions of these libraries indicated near equal incorporation levels of these modified dC and T residues (FIG. 4B).

### REFERENCES

1. Gramlich, P. M., Wirges, C. T., Gierlich, J. and Carell, T. (2008) Synthesis of modified DNA by PCR with alkyne-bearing purines followed by a click reaction. Org Lett, 10, 249-251.
2. Famulok, M. (1999) Oligonucleotide aptamers that recognize small molecules. Curr Opin Struct Biol, 9, 324-329.
3. Xu, W. and Ellington, A. D. (1996) Anti-peptide aptamers recognize amino acid sequence and bind a protein epitope. Proc Natl Acad Sci USA, 93, 7475-7480.
4. Tuerk, C. and Gold, L. (1990) Systematic evolution of ligands by exponential enrichment: RNA ligands to bacteriophage T4 DNA polymerase. 249, 505-510.
5. Daniels, D. A., Chen, H., Hicke, B. J., Swiderek, K. M. and Gold, L. (2003) A tenascin-C aptamer identified by tumor cell SELEX: systematic evolution of ligands by exponential enrichment. Proc Natl Acad Sci USA, 100, 15416-15421.
6. Shangguan, D., Li, Y., Tang, Z., Cao, Z. C., Chen, H. W., Mallikaratchy, P., Sefah, K., Yang, C. J. and Tan, W. (2006) Aptamers evolved from live cells as effective molecular probes for cancer study. Proc Natl Acad Sci USA, 103, 11838-11843.
7. Magalhaes, M. L., Byrom, M., Yan, A., Kelly, L., Li, N., Furtado, R., Palliser, D., Ellington, A. D. and Levy, M. (2012) A general RNA motif for cellular transfection. Mol Ther, 20, 616-624.
8. Jenison, R. D., Gill, S. C., Pardi, A. and Polisky, B. (1994) High-resolution molecular discrimination by RNA. Science, 263, 1425-1429.
9. Ni, X., Castanares, M., Mukherjee, A. and Lupold, S. E. (2011) Nucleic acid aptamers: clinical applications and promising new horizons. Curr Med Chem, 18, 4206-4214.
10. Gold, L., Ayers, D., Bertino, J., Bock, C., Bock, A., Brody, E. N., Carter, J., Dalby, A. B., Eaton, B. E., Fitzwater, T. et al. (2010) Aptamer-based multiplexed proteomic technology for biomarker discovery. PLoS One, 5, e15004.
11. Vaught, J. D., Bock, C., Carter, J., Fitzwater, T., Otis, M., Schneider, D., Rolando, J., Waugh, S., Wilcox, S. K. and Eaton, B. E. (2010) Expanding the chemistry of DNA for in vitro selection. J Am Chem Soc, 132, 4141-4151.
12. M, K., R, Y., K, M., S, Y. and I, H. (2103) Generation of high-affinity DNA aptamers using an expanded genetic alphabet. Nat Biotechnol, 31, 453-457.
13. Cho, M., Xiao, Y., Nie, J., Stewart, R., Csordas, A. T., Oh, S. S., Thomson, J. A. and Soh, H. T. (2010) Quantitative selection of DNA aptamers through microfluidic selection and high-throughput sequencing. Proc Natl Acad Sci USA, 107, 15373-15378.
14. Schutze, T., Wilhelm, B., Greiner, N., Braun, H., Peter, F., Morl, M., Erdmann, V. A., Lehrach, H., Konthur, Z., Menger, M. et al. (2011) Probing the SELEX process with next-generation sequencing. PLoS One, 6, e29604.
15. Kupakuwana, G. V., Crill, J. E., 2nd, McPike, M. P. and Borer, P. N. (2011) Acyclic identification of aptamers for human alpha-thrombin using over-represented libraries and deep sequencing. PLoS One, 6, e19395.
16. Hoon, S., Zhou, B., Janda, K. D., Brenner, S. and Scolnick, J. (2011) Aptamer selection by high-throughput sequencing and informatic analysis. Biotechniques, 51, 413-416.
17. Sakthivel, K. and Barbas, C. F. (1998) Expanding the potential of DNA for binding and catalysis: Highly functionalized dUTP derivatives that are substrates for thermostable DNA polymerases. Angew Chem Int Edit, 37, 2872-2875.
18. Kiessling, L. L., Gestwicki, J. E. and Strong, L. E. (2006) Synthetic multivalent ligands as probes of signal transduction. Angew Chem Int Ed Engl, 45, 2348-2368.
19. Kiessling, L. L., Gestwicki, J. E. and Strong, L. E. (2000) Synthetic multivalent ligands in the exploration of cell-surface interactions. Curr Opin Chem Biol, 4, 696-703.
20. Lee, S. E., Sidorov, A., Gourlain, T., Mignet, N., Thorpe, S. J., Brazier, J. A., Dickman, M. J., Hamby, D. P., Grasby, J. A. and Williams, D. M. (2001) Enhancing the catalytic repertoire of nucleic acids: a systematic study of linker length and rigidity. Nucleic Acids Res, 29, 1565-1573.
21. Long, S. B., Long, M. B., White, R. R. and Sullenger, B. A. (2008) Crystal structure of an RNA aptamer bound to thrombin. RNA, 14, 2504-2512.
22. Andrus, A. and Kuimelis, R. G. (2001) Base composition analysis of nucleosides using HPLC. Curr Protoc Nucleic Acid Chem, Chapter 10, Unit 10-16.

1. An oligonucleotide comprising a nucleotide residue comprising a modified nucleobase, wherein the modified nucleobase is a pyrimidine modified at the 5 position thereof, or a purine modified at the 7 position thereof.

2. The oligonucleotide of claim 1, wherein the modified nucleobase is a pyrimidine modified at the 5 position thereof with one of the following: 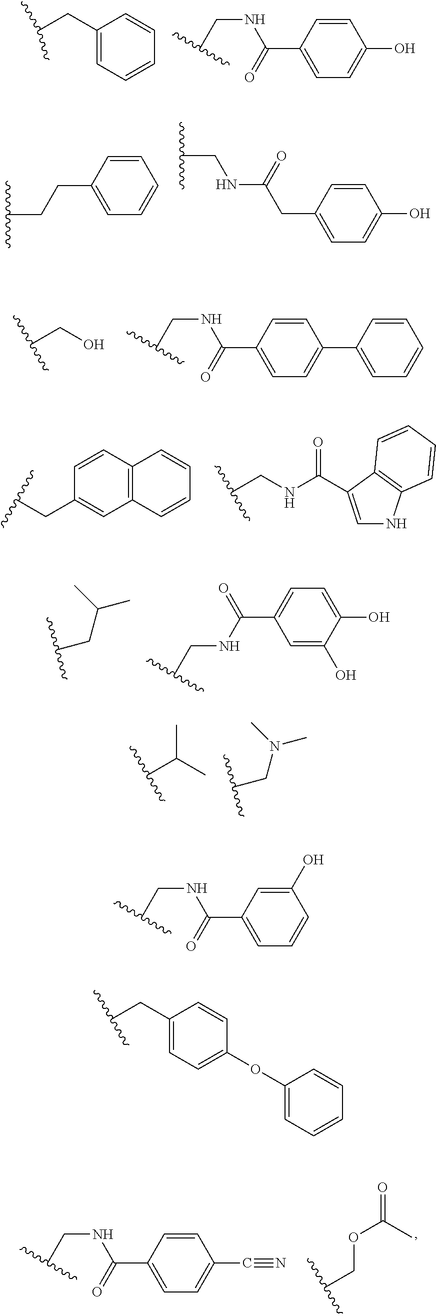
or wherein the modified nucleobase is a purine modified at the 7 position thereof with one of the following: 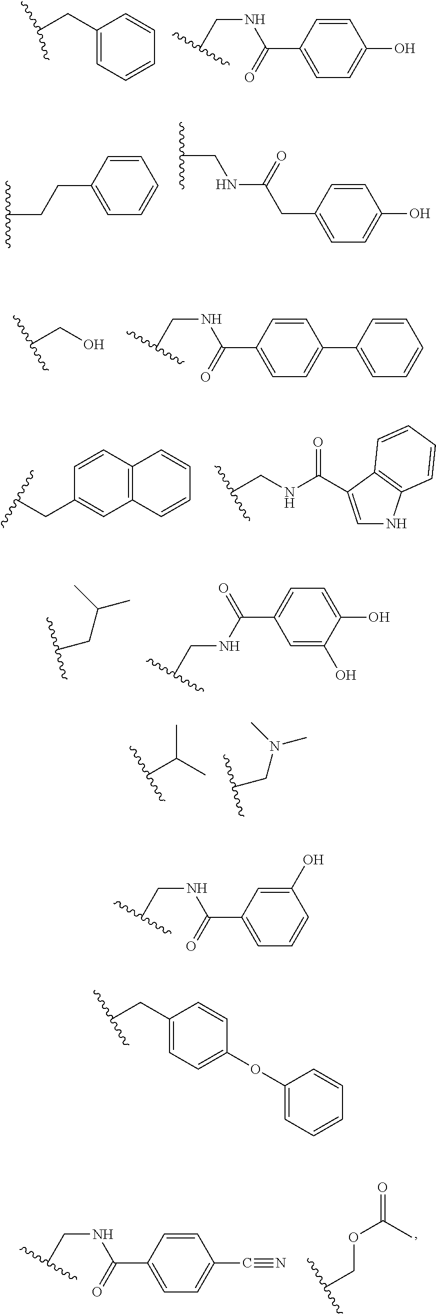
wherein the wavy line in the structures represents the point of attachment of the modifying group to the base of the modified nucleotide residue.

3. The oligonucleotide of claim 1, wherein the modifying group is attached via an alkyne to the base of the modified nucleotide residue.

4. The oligonucleotide of claim 1, wherein the nucleotide residue comprising a modified nucleobase comprises a deoxyuridine or a deoxycytidine or a deoxyadenine or a deoxyguanosine.

5. The oligonucleotide of claim 1, wherein the nucleotide residue comprising a modified nucleobase comprises one of the following structures: 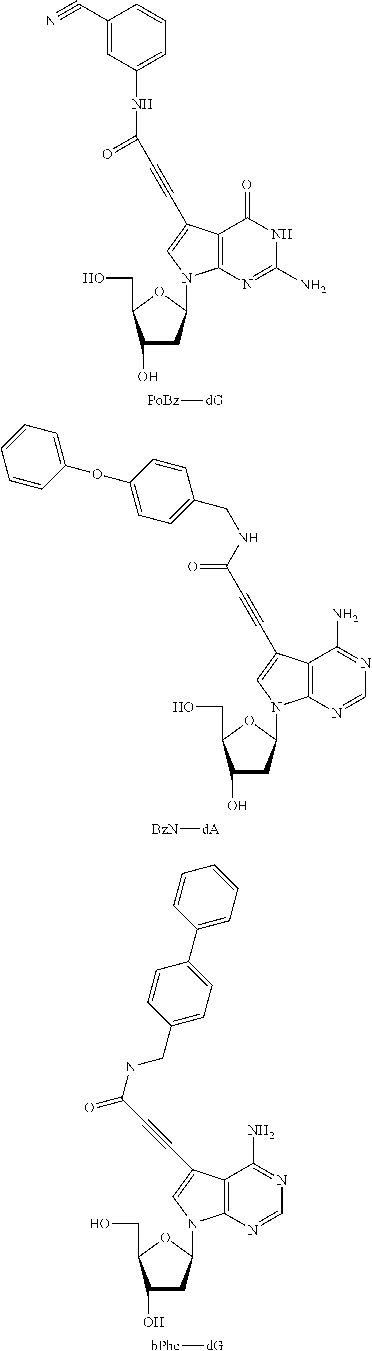 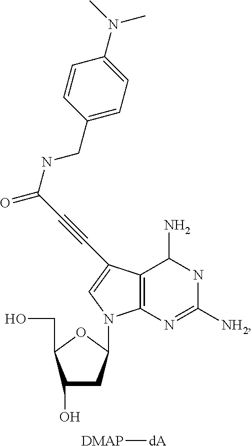
wherein each of the OH groups on the deoxyribose are, optionally, replaced with an internucleotide phosphodiester bond when the residue is not a terminal residue within the oligonucleotide.

6. The oligonucleotide of claim 1, wherein the nucleotide residue comprising a modified nucleobase comprises one of the following structures: 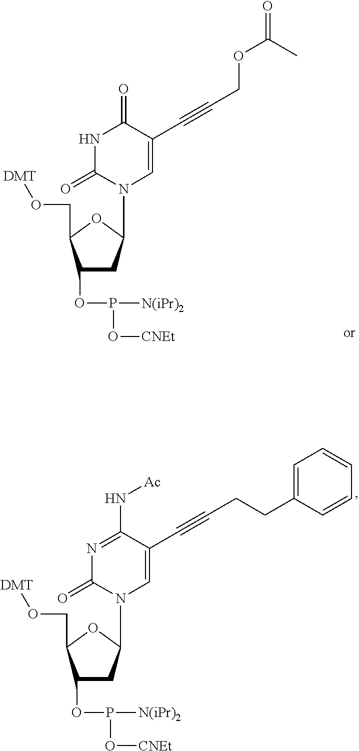
wherein each of the DMT and CNEt groups on the deoxyribose are, optionally, replaced with a further nucleotide, via an internucleotide phosphodiester bond, when the residue is not a terminal residue within the oligonucleotide.

7. The oligonucleotide of claim 1, comprising more than one nucleotide residue comprising a modified nucleobase, wherein the modified nucleobases are each independently chosen from: a pyrimidine modified at the 5 position thereof and a purine modified at the 7 position thereof.

8. The oligonucleotide of claim 1, comprising at least two different modified nucleobases.

9. The oligonucleotide of claim 1, comprising at least three different modified nucleobases.

10. (canceled)

11. The oligonucleotide of claim 1, further comprising a predefined ligand, for a protein target, attached thereto.

12. The oligonucleotide of claim 11, wherein the predefined ligand is a low-affinity ligand for the protein target.

13. The oligonucleotide of claim 12, wherein the low-affinity ligand is a glycan.

14. The oligonucleotide of claim 11, comprising the following residue: 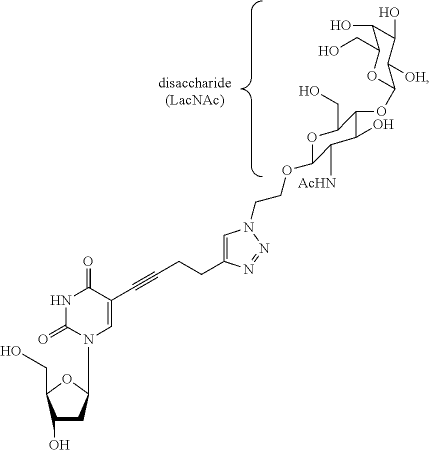 [SMILES: CC(=O)NC1C(OCCn2cc(CCC#Cc3cn([C@H]4C[C@@H](O)[C@@H](CO)O4)c(=O)[nH]c3=O)nn2)OC(CO)C(OC2OC(CO)C(O)C(O)C2O)C1O]
wherein each of the OH groups on the deoxyribose are, optionally, replaced with an internucleotide phosphodiester bond when the residue is not a terminal residue within the oligonucleotide.

15. The oligonucleotide of claim 1, wherein the predefined ligand for a protein target is attached through a functional group attached to a nitrogenous base of a nucleotide thereof.

16. (canceled)

17. The oligonucleotide of claim 1, wherein the oligonucleotide comprises (a) (i) a 5′ non-random region contiguous at its 3′ end with (ii) a random region contiguous at its 3′ end with (iii) a 3′ non-random region; or (b) (i) a 5′ non-random region contiguous at its 3′ end with (ii) a random region contiguous at its 3′ end with (iii) a second nonrandom region contiguous at its 3′ end with (iv) a second random region contiguous at its 3′ end with (v) a 3′ non-random region.

18. The oligonucleotide of claim 17, comprising one or more primer attachment sequences in a non-random region thereof.

19. The oligonucleotide of claim 18, wherein the one or more primers are universal primers.

20. The oligonucleotide of claim 18, comprising one or two double-stranded regions composed of intra-oligonucleotide base-pairing.

21. A method for identifying a ligand for a protein target comprising contacting the protein target with a plurality of oligonucleotides of claim 1, wherein at least two of the oligonucleotides have different sequences, subsequently washing the protein target to remove any unbound oligonucleotides of the plurality of oligonucleotides, recovering and sequencing oligonucleotides bound to the target protein, so as to thereby identify from the plurality of oligonucleotides one or more ligands for the protein target.

22. The method of claim 21, further comprising counting the number of oligonucleotides of a single sequence type recovered and sequenced, wherein an oligonucleotide with the greatest count is identified as the most efficacious ligand for the protein target.

23-42. (canceled)
